(19) United States
(12) Patent Application Publication
(10) Pub. No.: US 2008/0277264 A1
(43) Pub. Date: Nov. 13, 2008

(54) ALCOHOL PRODUCTION USING HYDRAULIC CAVITATION

(75) Inventor: Allison Sprague, Prescott (CA)

Correspondence Address: WOOD, HERRON & EVANS, LLP, 2700 CAREW TOWER, 441 VINE STREET, CINCINNATI, OH, 45202, US

(73) Assignee: FLUID-QUIP, INC., Springfield, OH (US)

(21) Appl. No.: 11/746,980

(22) Filed: May 10, 2007

(51) Int. Cl. C07C 29/00; C02F 1/36

(52) U.S. Cl. 204/157.9; 210/542

(57) ABSTRACT

A system and method is provided which includes using a liquid treatment apparatus, which is equipped with cyclonettes, for example, to subject a liquid medium processing stream in an alcohol production process to hydraulic cavitation, i.e., to shear under vacuum, at one or more locations. The liquid treatment apparatus, in one embodiment, is directed to the formation of a central axial jet and a vacuum chamber that can be sealed by the exiting jet. Cavitation is generated by directing a high velocity jet of liquid medium processing stream through a volume of vapor under a vacuum created in the chamber through which the jet travels. This can reduce the production cost of alcohol, such as ethanol, by improving alcohol yield per bushel, among other benefits. In one embodiment, the alcohol production process is a dry grind process, a modified dry grind process, or a wet mill process. In one embodiment the alcohol production process utilizes grain as a starting material.

### FIELD OF THE INVENTION

The present invention relates generally to alcohol production, and, more particularly, to using a liquid treatment apparatus to subject a liquid medium processing stream in an alcohol production process to hydraulic cavitation.

### BACKGROUND OF THE INVENTION

One alcohol of great interest today is ethanol, which can be produced from virtually any type of grain, but is most often made from corn. The U.S. ethanol industry has been producing record amounts of ethanol, all of which is being produced from about 74 ethanol plants located mainly within the corn-belt. Since its inception, the national market for fuel ethanol has grown from about 6.6 million liters (about 175 million gallons (gal)) in 1980 to greater than 7.9 billion liters (about 2.1 billion gal). Ethanol production could grow to approximately 1.9 trillion liters (approximately five (5) billion gal) by 2012. Consequently, ethanol producers are seeking methods to improve yields before incurring the high capital costs of direct plant expansion. Because of the ongoing need for ethanol, as well as recent and expected future rapid growth of the ethanol industry, producers are finding it difficult to incur the time and expense required to refine existing technologies to meet the potentially mandated increases and also remain cost competitive with intense ethanol producer competition. Higher yields are also desired for other types of alcohol.

The methods for producing various types of alcohol from grain generally follow similar procedures, depending on whether the process is operated wet or dry. Work in the field has included generation of cavitation, which involves the formation of vapor bubbles that upon collapse can cause the dissolution of water into hydroxyl radicals, such as by the formation of shock waves to physically modify process streams. Such cavitation can be induced by electrically driven transducers (sonication), for example. The goal of cavitation processing is to generate many fine bubbles, which upon their implosion, create intense, but highly localized temperatures and pressures. This energy release then causes dissolution of water molecules and the creation of free hydroxyl radicals. Along with sonication, the patent literature discloses a multitude of methods and apparatuses for generating cavitation. However, the inefficiency of the known methods and apparatuses is understood to have restricted commercial acceptance.

Thus, there remains the apparent conundrum of highly effective methods of alcohol production, such as ethanol production, utilizing cavitation which increase yields but at an energy cost that thwarts widespread implementation.

### SUMMARY OF THE INVENTION

In an embodiment of the present invention, a system is provided including a liquid treatment apparatus such as equipped with cyclonettes, as further described below, for generating cavitation by hydraulic means, i.e., hydraulic cavitation, and an alcohol production facility having a liquid medium processing stream, the alcohol production facility being adapted for use with the liquid treatment apparatus. In one embodiment, the alcohol production facility is an ethanol production facility. In one embodiment, the ethanol production facility utilizes a dry grind process, modified dry grind process, or wet mill process. In one embodiment, the ethanol production facility utilizes grain as a starting material. In one embodiment, the grain is corn, sorghum, wheat, barley, oats, or rice. The liquid medium processing stream can include heavy steep water, an uncooked slurry, a cooked mash, a liquefied mash, and (for a dry grind process) whole stillage, thin stillage and wet cake.

In another embodiment of the present invention, a method is also provided which includes using a liquid treatment apparatus to subject the liquid medium processing stream in an alcohol production process to hydraulic cavitation, i.e., shear under vacuum, at one or more locations. In one embodiment, the liquid treatment apparatus is provided with a plurality of cyclonettes. In one embodiment, the alcohol production process is an ethanol production process. In one embodiment, the ethanol production process is a dry grind process, a modified dry grind process, or a wet mill process. In one embodiment the ethanol production process utilizes grain as a starting material. In one embodiment, the grain is corn, sorghum, wheat, barley, oats, or rice. The liquid medium processing stream can include heavy steep water, an uncooked slurry, a cooked mash, a liquefied mash, and (for a dry grind process) whole stillage, thin stillage and wet cake.

Although the systems and methods described herein focus primarily on ethanol production primarily from corn, it should be noted that any of the systems and methods described can be used in other types of alcohol production facilities and with other types of grain feedstock. The various embodiments provide systems and methods for improving alcohol production, such as ethanol production, using the liquid treatment apparatus, which is explained in detail further below. The particular improvement achieved depends on several factors, including, but not limited to, the type of alcohol being produced, the particular point(s) in the process at which the liquid treatment apparatus is used, and so forth. Other factors particular to the operation can also affect the benefit obtained. These include, but are not limited to, the flow rate of the fluid medium, the nature of the medium to be acted upon, including type and amount of particulate content, temperature, and so forth.

In one embodiment, ethanol fermentation speed and/or ethanol yields can be increased by using the liquid treatment apparatus to generate hydraulic cavitation in a dry grind, modified dry grind, or wet mill ethanol production process.

In one embodiment, the amount of chemical and biological additives used can be decreased by using the liquid treatment apparatus to generate hydraulic cavitation in a dry grind, modified dry grind, or wet mill ethanol production process at one or more points prior to the fermentation step.

In one embodiment, energy costs may be reduced by using the liquid treatment apparatus to generate hydraulic cavitation prior to and/or after cooking in a dry grind, modified dry grind, or wet mill ethanol production process. As a result, key processes, such as jet cooking can either be completed at lower temperatures, at higher solids concentrations and/or shorter durations, or be eliminated altogether.

In one embodiment, transgenic proteins and transgenic nucleic acids of genetically modified feedstocks can be denatured or degraded by using the liquid treatment apparatus to generate hydraulic cavitation at one or more points in a dry grind, modified dry grind, or wet mill ethanol production process. As a result, stringent export requirements limiting or forbidding the shipment of genetically modified food and feed products, can be met.

In one embodiment, bacteria and/or fungi and/or yeast contaminants can be rendered nonviable by using the liquid treatment apparatus to generate hydraulic cavitation in a dry grind, modified dry grind, or wet mill ethanol production process just prior to the fermentation step. As a result, infection of the product during fermentation is reduced or prevented.

In one embodiment, complex proteins (i.e., proteins not normally bio-available to the digestive systems of many animals, i.e., proteins not susceptible to hydrolysis to amino acids by proteolytic enzymes) present in whole stillage may be broken down by using the liquid treatment apparatus to generate hydraulic cavitation, producing animal feeds having proteins which can be less complex and therefore more bio-available to the digestive systems of many animals.

In one embodiment, the insoluble solids in whole stillage may be sheared, i.e., homogenized, resulting in increased surface area of the solids, which reduces drying time downstream.

Embodiments of the invention further include a method for increasing fermentable starch levels in a dry grind alcohol production process having a liquid medium processing stream including subjecting the liquid medium processing stream to one or more liquid treatment apparatuses equipped with cyclonettes for generating hydraulic cavitation, wherein alcohol yield is increased and residual starch levels reduced. In one embodiment, the alcohol production process is a dry grind ethanol production process, further wherein ethanol yield is increased. In one embodiment, the ethanol production process also produces distiller's dry grain solids containing the residual starch and protein. In one embodiment, cell macromolecules can be stripped away from starch granule surfaces.

In one embodiment, the cell macromolecules are protein, fiber cellulose and fiber hemicellulose. In one embodiment, gelatinized starch granules present in the liquid medium processing stream can be broken open or disintegrated further wherein availability of gelatinized starch granules to enzymes added to the liquid medium processing stream is increased during liquefaction and saccharification.

Embodiments of the present invention further include a system having one or more liquid treatment apparatuses, such as equipped with the cyclonettes, for example, to generate hydraulic cavitation and an ethanol production facility having a corn-based liquid medium processing stream, the ethanol production facility adapted for use with the liquid treatment apparatus(es), wherein hydraulic cavitation is applied to the corn-based liquid medium processing stream in one or more locations. Embodiments of the invention further include a method of applying hydraulic cavitation, via the liquid treatment apparatus, to a corn-based liquid medium processing stream in an ethanol production process in one or more locations. In one embodiment, the ethanol production facility utilizes a wet mill process and the liquid treatment apparatus applies hydraulic cavitation to the liquid medium processing stream before a fiber washing step. In one embodiment, the ethanol production facility utilizes a dry grind process and the liquid treatment apparatus applies hydraulic cavitation to the liquid medium processing stream prior to fermentation. In one embodiment, the liquid treatment apparatus applies hydraulic cavitation to the liquid medium processing stream before or after a jet cooking step.

In one embodiment of the present invention, the liquid treatment apparatus utilized is directed to the formation of a central axial jet and a vacuum chamber that can be sealed by the exiting jet. Thus, cavitation is generated by directing a high velocity jet of fluid, or liquid medium processing stream, for example, through a volume of vapor under a vacuum created in the chamber through which the jet travels. In other words, the liquid medium processing stream is subjected to shear under vacuum to generate hydraulic cavitation. Also, turbulence is induced within the jet to create vortices that under vacuum provide nucleation sites for the formation of additional vapor bubbles.

To that end, the liquid treatment apparatus employs a high-speed jet of liquid medium processing stream, flowing axially and centrally through a chamber to generate a vacuum within a confined space. In one embodiment, the liquid treatment apparatus includes the provision of a liquid-free volume around the jet near the inlet end of the chamber to cause vapor to accumulate. The discharge opening of the chamber is designed so that it will be completely filled by the exiting jet of fluid, so as to seal the chamber and permit maintenance of a vacuum.

In one embodiment, conventional hydrocyclone apparatuses may be modified and, thus, adapted to the aforementioned configuration for generating hydraulic cavitation. For example, a conventional cyclonette may be employed to provide a central axial jet with its conventional, tangential feed opening blocked. Additionally, a multiplicity of cyclonettes may be mounted in a housing, essentially as shown in U.S. Pat. No. 5,388,708, but with the cyclonettes fed from the annular, outer chamber and discharging into the inner or central cylindrical chamber. There are a number of advantages to this arrangement. First, because the discharge jets are directed towards one another, the velocity head of the jets is converted to pressure head. This causes the vapor bubbles to collapse asymmetrically because the pressure on one side of the bubbles is greater than on the other, which results in the formation of high speed liquid jets that can be physically disruptive. Second, the collapse of the bubbles tends to occur at the center of the chamber, well away from the walls. This results in reduced cavitation damage to the housing. Third, when bubbles collapse, shock waves are generated. As these shock waves propagate in a radial direction, the shock wave energy projected on the cross sectional area of another bubble causes pressure variations within the bubble. This tends to generate heat within the bubble, which can increase chemical reaction rates within and around the bubble. It may also cause the bubble to collapse asymmetrically, creating the aforementioned high speed liquid jets. As a result, there is a synergistic effect to having multiple bubbles collapse in close proximity. The proposed arrangement of multiple cavitation generators mounted in a housing so the discharges flow in a radial direction towards a common center tends to optimize this effect.

Alternatively, the tangentially directed inlet port in the cyclonettes of the '708 patent may be employed to inject a second liquid medium processing stream into the cyclonette along its inside wall in a spiral flow path. Vapor within the cyclonette will tend to be dragged axially toward the discharge end by the linear jet and in a spiral path by the second liquid medium processing stream. When the two high-velocity streams approach one another, the shear created due to the differences in velocity will tend to create a turbulent mixing zone that will disrupt the vapor film and generate bubbles. Increasing the fluid velocities will increase shear and reduce the size of the bubbles. It will also result in increased vacuum within the chamber and the generation of more vapor.

With this design, cavitation can be maintained at very low inlet fluid pressure—on the order of about 30 psi or less, for example, with liquid at about 10° C. and atmospheric pressure discharge. Also, the high shear generated helps reduce bubble size, which in turn, increases bubble surface to volume ratio and improves chemical reaction rates. As long as the velocity head of the fluid exiting the chamber exceeds the static pressure in the discharge zone, a vacuum will be generated within the chamber. Once pressure within the chamber drops to the vapor pressure of the liquid, vapor is generated around the inlet jet, and at locations of high turbulence within the jet, and cavitation occurs. Thus, the amount of vapor entrained can be almost independent of pressure in the discharge zone.

As a modification of this embodiment, the main inlet jet may pass through a vortex finder of conventional design, except that, in addition to the flow being directed into the cyclonette from the vortex finder (instead of out of the cyclonette through the vortex finder), the vortex finder is modified to impart a spin to the incoming jet in a direction opposite to the direction of the tangential inlet flow. The result is that the collision of the two streams flowing in opposite directions creates a shear on the vapor trapped between the two streams that shears the vapor film into tiny bubbles, leading to increased cavitation efficiency.

In still a further modification of the liquid treatment apparatus, the enhancement of fine bubble generation may be attained by the interposition in the flow path into the cyclonette of a washer-shaped orifice plate. The abrupt decrease in diameter of the flow path through a modified vortex finder, not only accelerates flow and decreases pressure, but generates an intense shear zone within the jet that leads to the formation of a virtual fog of tiny bubbles, the collapse of which, generates localized extreme temperatures and pressures.

Accordingly, the systems and methods of the present invention utilize one or more liquid treatment apparatuses, such as equipped with cyclonettes, for example, for subjecting the liquid medium processing stream, in alcohol production, to hydraulic cavitation. This can reduce the production cost of alcohol, such as ethanol, by improving alcohol yield per bushel, reducing processing times for higher throughput, reducing operating costs, and increasing the marketability of co-products, among other benefits.

### BRIEF DESCRIPTION OF THE DRAWINGS

FIG. 1 is a diagram of a prior art method of ethanol production using a dry grind process;

FIG. 2 is a diagram of a prior art method of ethanol production using a modified dry grind process;

FIG. 3 is a diagram of a prior art method of ethanol production using a wet mill process;

FIG. 4 is an elevational view, partly in section, displaying an array of cyclonettes modified to generate hydraulic cavitation;

FIG. 5 is an elevational view of the extreme lower end of the device of FIG. 4 and with the cooperating inlet and outlet flow manifolds;

FIG. 6 is a cross-sectional view of a portion of FIG. 4 showing in greater detail the positioning of a modified cyclonette;

FIG. 7 is a horizontal view in cross-section taken along line 7-7 of FIG. 4;

FIG. 8 is a view similar to FIG. 7, but with portions removed to show the physical relationships of modified cyclonettes within an array with respect to each other;

FIG. 9 is an enlarged cross-sectional view of a cyclonette and vortex finder;

FIG. 10 is a view similar to FIG. 9, but showing a modified cyclonette and a modified vortex finder, together with an orifice plate;

FIG. 11 is a view similar to FIG. 10, but showing the flow of the liquid through the modified cyclonette, vortex finder and orifice plate;

FIG. 11A is a diagrammatic view of the liquid flow at point 11A in FIG. 11 and showing individual bubbles generated as the liquid flows through the inlet plate;

FIG. 11B is a view similar to FIG. 11A, but depicting the flow and bubbles at point 11B in FIG. 11 of the drawings;

FIG. 11C is a view similar to FIGS. 11A and 11B, but showing the individual bubbles somewhat dispersed at point 11C in FIG. 11 downstream of points 11A and 11B in FIG. 11;

FIG. 12 is a view similar to FIG. 9, but showing a modified flow path through the body of a cyclonette;

FIG. 13 is a view similar to FIG. 12, but with the extension of the vortex finder removed; and

FIG. 14 is a view similar to FIG. 10, but showing the orifice plate positioned downstream from the position shown in FIG. 10, closer to the throat area of the modified cyclonette;

FIG. 15 is a diagram showing one embodiment of a method of ethanol production using the liquid treatment apparatus to generate hydraulic cavitation in a dry grind process;

FIG. 16 is a diagram showing one embodiment of a method of ethanol production using the liquid treatment apparatus to generate hydraulic cavitation in a modified dry grind process; and

FIG. 17 is a diagram showing one embodiment of ethanol production using the liquid treatment apparatus to generate hydraulic cavitation in a wet mill process.

### DESCRIPTION OF THE PREFERRED EMBODIMENTS

In the following detailed description of embodiments of the invention, reference is made to the accompanying drawings that form a part hereof, and in which is shown by way of illustration specific preferred embodiments in which the subject matter may be practiced. These embodiments are described in sufficient detail to enable those skilled in the art to practice them, and it is to be understood that other embodiments may be utilized and that mechanical, chemical, structural, electrical, and procedural changes may be made without departing from the spirit and scope of the present subject matter. The following detailed description is, therefore, not to be taken in a limiting sense, and the scope of embodiments of the present invention is defined only by the appended claims.

The Detailed Description that follows begins with a discussion on the various known methods of ethanol production followed by a discussion of the liquid treatment apparatus useful herein for generating hydraulic cavitation of the liquid medium processing stream. This is followed by a detailed description of specific embodiments of the invention.

Ethanol Production Methods

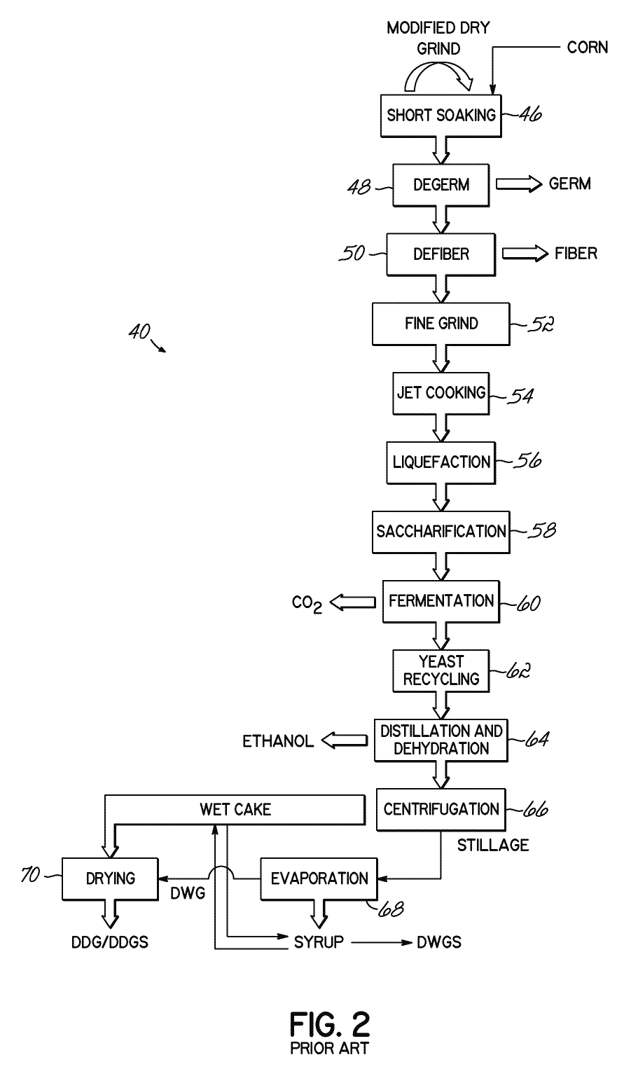

Virtually all of the fuel ethanol in the United States is produced from a wet mill process or a dry grind ethanol process. A newer process, known as a “modified” dry grind ethanol process is described below and shown in FIG. 2. Although virtually any type and quality of grain can be used to produce ethanol, the feedstock for these processes is typically a corn known as “No. 2 Yellow Dent Corn.” The “No. 2” refers to a quality of corn having certain characteristics as defined by the National Grain Inspection Association, as is known in the art. “Yellow Dent” refers to a specific type of corn as is known in the art. Sorghum grain is also utilized to a very small extent. Generally speaking, the current industry average for ethanol yield for both dry grind and wet mill plants is approximately 10.2 liters (approximately 2.7 gal) of ethanol produced per 25.4 kg (one (1) bushel) of No. 2 Yellow Dent Corn.

Dry grind ethanol plants convert corn into two products, namely ethanol and distiller's grains with solubles. If sold as wet animal feed, distiller's wet grains with solubles is referred to as DWGS. If dried for animal feed, distiller's dried grains with solubles is referred to as DDGS. In the standard dry grind ethanol process, one bushel of corn yields approximately 8.2 kg (approximately 18 lbs) of DDGS in addition to the approximately 10.2 liters (approximately 2.7 gal) of ethanol. This co-product provides a critical secondary revenue stream that offsets a portion of the overall ethanol production cost.

Wet mill corn processing plants convert corn grain into several different co-products, such as germ (for oil extraction), gluten feed (high fiber animal feed), gluten meal (high protein animal feed), and starch-based products such as ethanol, high fructose corn syrup, or food and industrial starch.

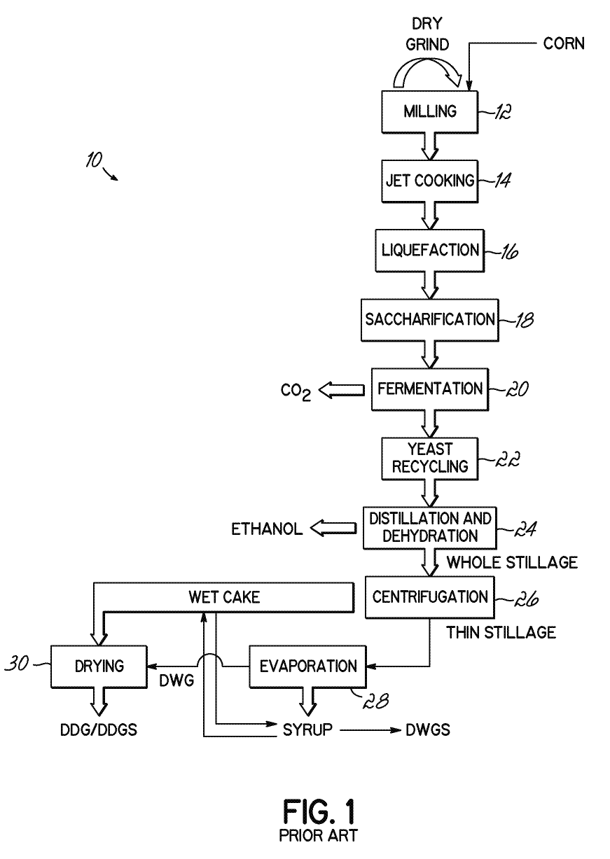

FIGS. 1-3 are flow diagrams of prior art ethanol production processes, such processes are fully discussed in U.S. Pat. No. 7,101,691, titled “Alcohol Production Using Sonication”, which is expressly incorporated by reference herein in its entirety.

FIG. 1 is a flow diagram of a prior art dry grind process 10. The process 10 begins with a milling step 12 in which dried whole corn kernels are passed through hammer mills, in order to grind them into meal or a fine powder. The ground meal is mixed with water to create a slurry, and a commercial enzyme called alpha-amylase is added (not shown). This slurry is then heated to approximately 120° C. for about 0.5 to three (3) minutes in a pressurized jet cooking process 14 in order to gelatinize (solubilize) the starch in the ground meal. Jet cooking refers to a cooking process performed at elevated temperatures and pressures, although the specific temperatures and pressures can vary widely. Typically, jet cooking occurs at a temperature of about 120 to 150° C. (about 248 to 302° F.) and a pressure of about 8.4 to 10.5 kg/cm² (about 120 to 150 lbs/in²), although the temperature can be as low as about 104 to 107° C. (about 220 to 225° F.) when pressures of about 8.4 kg/cm² (about 120 lbs/in²) are used. (This is in contrast to a non-jet cooking process, which refers to a process in which the temperature is less than the boiling point, such as about 90 to 95° C. (about 194 to 203° F.) or lower, down to about 80° C. (176° F.). At these lower temperatures, ambient pressure would be used).

This is followed by a liquefaction step 16 at which point additional alpha-amylase may be added. Liquefaction occurs as the mixture, or “mash” is held at 90 to 95° C. in order for alpha-amylase to hydrolyze the gelatinized starch into maltodextrins and oligosaccharides (chains of glucose sugar molecules) to produce a liquefied mash or slurry. In the embodiment shown in FIG. 1, this is followed by separate saccharification and fermentation steps, 18 and 20, respectively, although in most commercial dry grind ethanol processes, saccharification and fermentation occur simultaneously. This step is referred to in the industry as “Simultaneous Saccharification and Fermentation” (SSF). In the saccharification step 18, the liquefied mash is cooled to about 50° C. and a commercial enzyme known as gluco-amylase is added. The gluco-amylase hydrolyzes the maltodextrins and short-chained oligosaccharides into single glucose sugar molecules to produce a liquefied mash, which is also a “fermentation feed” when SSF is employed. In the fermentation step 20 a common strain of yeast (Saccharomyces cerevisae) is added to metabolize the glucose sugars into ethanol and CO₂. Both saccharification and SSF can take as long as about 50 to 60 hours. Upon completion, the fermentation mash (“beer”) will contain about 17% to 18% ethanol (volume/volume basis), plus soluble and insoluble solids from all the remaining grain components. Yeast can optionally be recycled in a yeast recycling step 22. In some instances the CO₂ is recovered and sold as a commodity product.

Subsequent to the fermentation step 20 is a distillation and dehydration step 24 in which the beer is pumped into distillation columns where it is boiled to vaporize the ethanol. The ethanol vapor is condensed in the distillation columns, and liquid alcohol (in this instance, ethanol) exits the top of the distillation columns at about 95% purity (190 proof). The 190 proof ethanol then goes through a molecular sieve dehydration column, which removes the remaining residual water from the ethanol, to yield a final product of essentially 100% ethanol (199.5 proof). This anhydrous ethanol is now ready to be used for motor fuel purposes.

Finally, a centrifugation step 26 involves centrifuging the residuals produced with the distillation and dehydration step 24, i.e., “whole stillage” in order to separate the insoluble solids (“wet cake”) from the liquid (“thin stillage”). The thin stillage enters evaporators in an evaporation step 28 in order to boil away moisture, leaving a thick syrup which contains the soluble (dissolved) solids from the fermentation. This concentrated syrup can be mixed with the centrifuged wet cake, and the mixture may be sold to beef and dairy feedlots as Distillers Wet Grain with Solubles (DWGS). Alternatively, the wet cake and concentrated syrup mixture may be dried in a drying step 30 and sold as Distillers Dried Grain with Solubles (DDGS) to dairy and beef feedlots.

FIG. 2 is a flow diagram of a prior art modified dry grind ethanol production process 40. The process 40 begins with a short soaking 46 of the corn for up to ten hours. The soaked corn is then degermed in a degerm step 48 and de-fibered in a defiber step 50. These processes physically remove and separate germ and coarse fiber, i.e., pericarp fiber from incoming whole kernel corn. (Coarse fiber or pericarp fiber is the outer covering of the corn kernel and is also referred to as “bran.” Coarse fiber can be mechanically separated and is obvious to the human eye, as opposed to fine fiber, i.e., cellular fiber embedded within the endosperm matrix, which is not easily mechanically separated due to its microscopic size and is not visible to the human eye). The remaining endosperm is then finely ground in a fine grind step 52 as shown. (This step takes the place of the hammer milling of whole, intact kernels, with the conventional dry grind process of FIG. 1). In the diagram shown in FIG. 2, the separated, finely ground endosperm is processed in the same manner as with a conventional prior art dry grind ethanol process, which includes jet cooking 54, liquefaction 56, saccharification 58, fermentation 60, yeast recycling 62 (in some instances), distillation and dehydration 64, centrifugation 66, evaporation 68 and drying 70 as described above in FIG. 1. The “stillage” produced after centrifugation 66 in the modified dry grind process 40 is often referred to as “whole stillage” although it technically is not the same type of whole stillage produced with the dry grind process described in FIG. 1, since no insoluble solids are present. Others skilled in the art may refer to this type of stillage as “thin” stillage.

The separated germ can be sold for corn oil extraction. The separated corn fiber can be fermented to produce ethanol in an alternate process, or can be extracted for higher value chemicals and nutraceuticals. Examples of chemicals and nutraceuticals extracted from corn fiber include fiber specialty oils, fiber phytosterols, fiber gums, fiber carotenoids, fiber tocopherols, and any other nutraceuticals and chemicals extracted from corn fiber. For a more detailed discussion of a prior art modified dry grind ethanol production process see, for example, U.S. Pat. No. 6,254,914 to Singh, et al., entitled, “Process for Recovery of Corn Coarse Fiber (Pericarp)”, issued Jul. 3, 2001 and U.S. Pat. No. 6,592,921 to Taylor, et al., entitled, “Method of Removing the Hull from Corn Kernels,” issued Jul. 15, 2003, both of which are incorporated herein by reference.

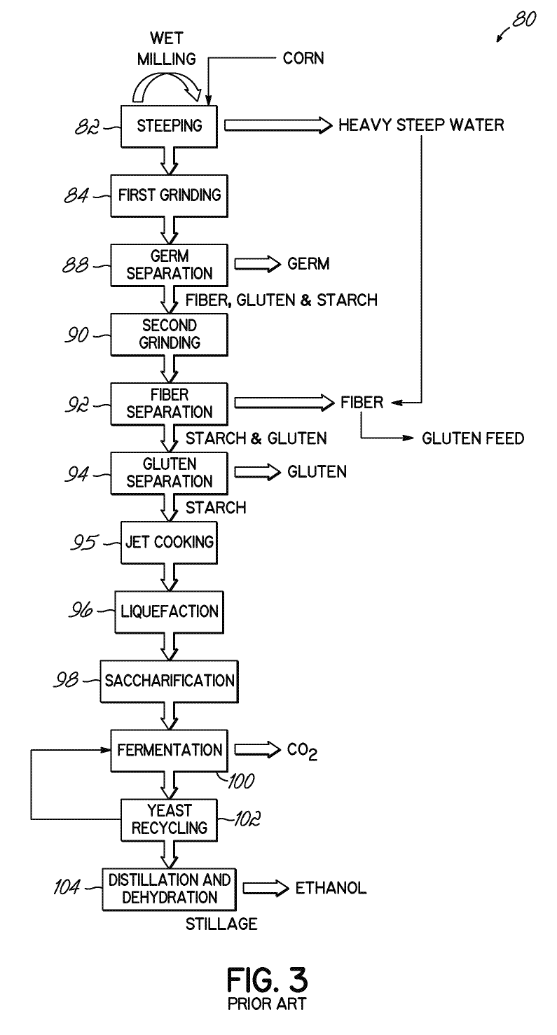

FIG. 3 is a flow diagram of a prior art wet mill ethanol production process 80. The process 80 begins with a steeping step 82 in which the corn is soaked for 24 to 48 hours in a solution of water and sulfur dioxide in order to soften the kernels for grinding, leach soluble components into the steep water, and loosen the protein matrix with the endosperm. The mixture of steeped corn and water is then fed to a degermination mill step (first grinding) 84 in which the corn is ground in a manner that tears open the kernels and releases the germ. This is followed by a germ separation step 88 that occurs by flotation and use of a hydrocyclone. The remaining slurry, which is now devoid of germ, but containing fiber, gluten (i.e., protein) and starch, is then subjected to a fine grinding step (second grinding) 90 in which there is total disruption of endosperm and release of endosperm components, namely gluten and starch, from the fiber. This is followed by a fiber separation step 92 in which the slurry is passed through a series of screens in order to separate the fiber from starch and gluten, and to wash the fiber clean of gluten and starch. This is followed by a gluten separation step 94 in which centrifugation or hydrocyclones separate starch from the gluten. As with the dry grind process described in FIG. 1, the resulting purified starch co-product then undergoes a jet cooking step 95. This is followed by liquefaction 96, saccharification 98, fermentation 100, yeast recycling 102 and distillation/dehydration 104. No centrifugation step is necessary at the end of the wet mill ethanol production process 80 as the germ, fiber and gluten have already been removed in the previous separation steps 88, 92 and 94. As with the modified dry grind process discussed in FIG. 2, the “stillage” produced after distillation and dehydration 104 in the wet mill process 80 is often referred to as “whole stillage” although it also is technically not the same type of whole stillage produced with the dry grind process described in FIG. 1, since no insoluble solids are present. Other wet mill producers may refer to this type of stillage as “thin” stillage.

Maximum theoretical ethanol yields in a commercial ethanol plant can only be as high as the total starch content of the corn feedstock. Most commercial ethanol plants do not achieve maximum theoretical ethanol yields. For example, with dry grind commercial ethanol plants, only “fermentable starch” is completely converted to ethanol, while the non-fermentable starch remains in the whole stillage at the end of fermentation. As an example, the DDGS produced from a standard dry grind ethanol process may contain as much as three (3) to 13% starch. This residual starch represents lost income in terms of inability of the ethanol plant to achieve maximum theoretical ethanol yield based on feedstock total starch content.

The inability to achieve substantially 100% conversion of starch to ethanol is due to several factors that are not fully understood. These factors include, but are not limited to, binding of starch granules to fine or coarse fiber (pericarp), binding of starch granules to protein bodies and protein matrices, very tight packing of starch granules, very tight binding of amyloplasts which contain starch granules, the internal molecular structure of the starch granules, which tends to make the starch “resistant” to gelatinization and enzymatic degradation, and the like.

Liquid Treatment Apparatus/Hydraulic Cavitation Technology

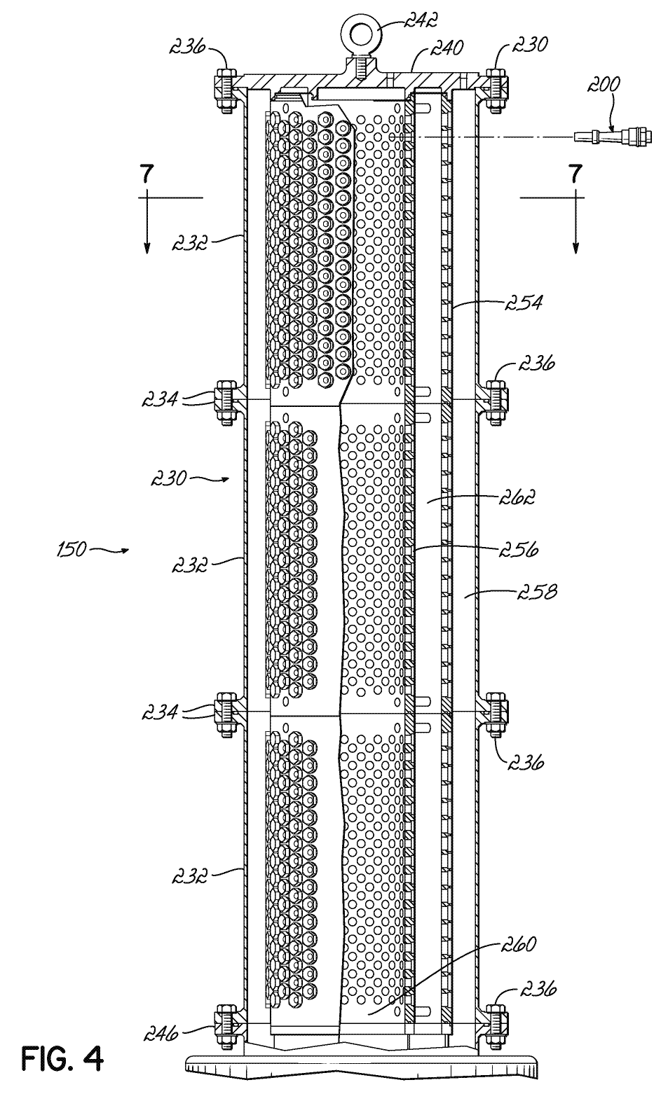

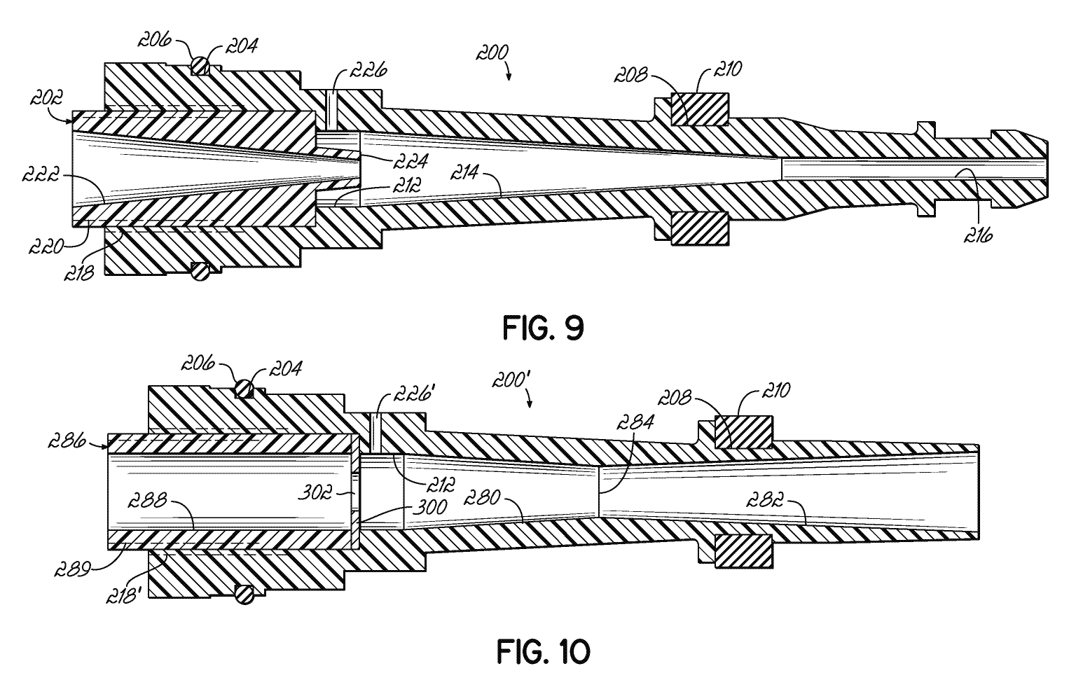

Turning now to embodiments of the liquid treatment apparatus 150 (FIG. 4) of the present invention, FIG. 9 shows a more or less conventional cyclonette 200 with a vortex finder 202 installed in the left hand end of the cyclonette. The left-hand end of the cyclonette may be provided with an annular groove 204 into which an O-ring 206 may be seated. To the right of the O-ring 206, as seen in FIG. 9, a second annular groove 208 may be formed to receive a second O-ring 210 of more or less rectangular cross-sectional configuration. Interiorly of the cyclonette 200, a flow path is provided comprising a throat portion 212, an inwardly tapering flow channel 214, and a terminal flow channel 216 of narrower constant diameter. At its left-hand end, as seen in FIG. 9, the cyclonette 200 may be provided with an internally threaded socket 218 receiving the complementary external threads 220 of the vortex finder 202. The vortex finder has a uniformly inwardly tapering wall 222 and an extension 224 projecting into the throat portion 212 of the cyclonette. Lastly, the cyclonette may be provided with a passageway 226 extending through a wall of the cyclonette 200 into the throat section 212.

With reference now to FIG. 4, a housing 230 is shown comprising cylinders 232, each having outwardly projecting annular flanges 234 to permit two or more cylinders 232 to be clamped together by bolts 236 to form a continuous, outer, annular chamber 258. While three cylinders 232 are shown in FIG. 4, it will be apparent that more or less cylinders may be employed, depending on the desired length of the annular outer chamber. At its upper end, the annular outer chamber is capped by a closure plate 240 having a lifting ring 242. The closure plate 240 is clamped to the upper end of the uppermost cylinder 232 in a manner similar to the clamping between adjacent cylinders by means of bolts 236.

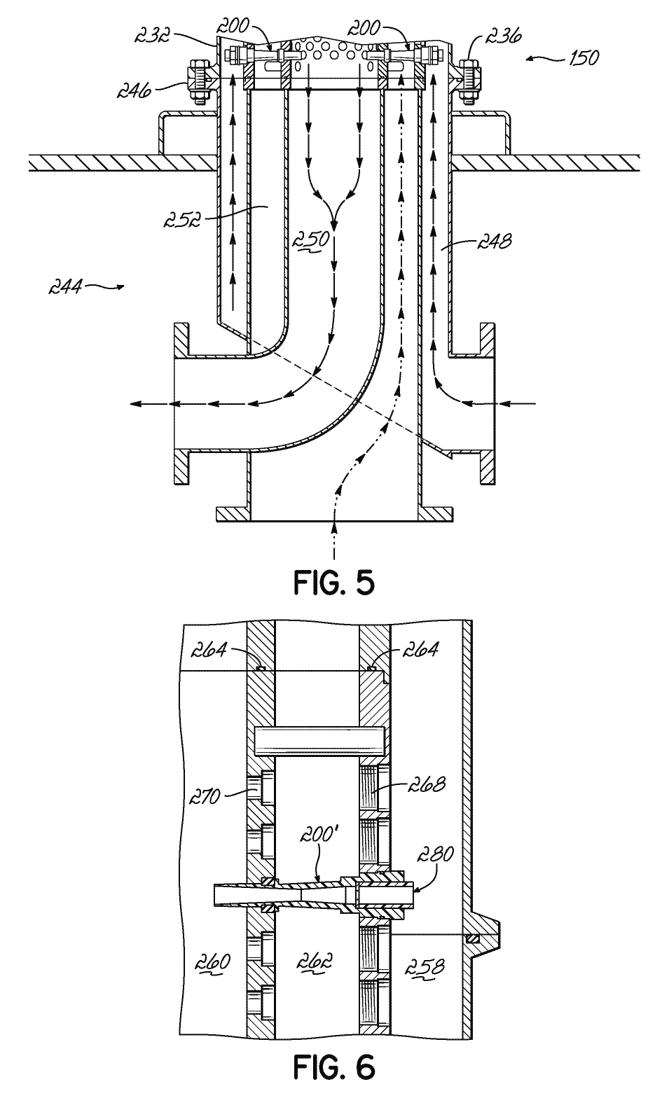

With reference now to FIGS. 4 and 5, it will be seen that the lowermost cylinder 232 is attached at its lower end by means of bolts 236 to a manifold system 244. At its upper end, the manifold system 244 has an outwardly projecting annular flange 246 to which the lower most cylinder 232 is clamped by the bolts 236 as shown in FIG. 5 of the drawings. The manifold system 244 comprises three concentric flow channels, namely, an outer feed channel 248, a central, outwardly-flowing channel 250, and an intermediate channel 252, which may or may not be used during the practice of the present invention, as will be described in more detail.

As seen in FIG. 4, positioned concentrically within the outer cylinders 232 are intermediate cylinders 254 and inner cylinders 256, which are each superimposed upon each other and clamped by the clamping action between the outer cylinders, the top plate 240 and the lower annular rim 246 of the manifold system 244. It will thus be apparent with reference to FIGS. 4 and 5 that the outer and intermediate cylinders form the annular outer chamber 258 communicating with the outer feed manifold 248, an inner or central chamber 260, communicating with the manifold 250, and an intermediate chamber 262 communicating with the manifold 252.

As best seen in FIG. 6, adjoining sets of intermediate and inner cylinders may be provided with annular grooves 264 and 266 to receive any convenient sealing means. Intermediate cylinders 254 are also provided with closely spaced openings 268 to receive cyclonettes that may be of more or less conventional design of a type shown in FIG. 9 or of various modified forms, which will be described presently in more detail. In any case, the cyclonettes are secured in any convenient manner in the openings 268 with the opposite ends of the cyclonettes being received in openings 270 in the cylinders 256. In FIG. 6, the openings 268 are shown as having internal threads, which could receive complementary external threads on the exterior of the cyclonettes. In this regard, O-rings, such as those shown at 206 and 210 in FIG. 9, may be utilized to create seals with the cylinders 254 and 256, respectively.

However, the particular manner of securing the cyclonettes in the intermediate and interior cylinders 254 and 256 does not form a part of the present invention, and any convenient means may be utilized. In any case, the positioning of a cyclonette, regardless of its specific configuration, in the manner shown in FIG. 6 permits the liquid delivered through the outer manifold 248 and into the annular outer chamber 258 to flow into an insert 286 and then into the upstream end of the cyclonette and out its downstream end where it is immersed in the liquid being treated, which is being collected in the inner or central cylindrical chamber 260 and then out through the manifold 250.

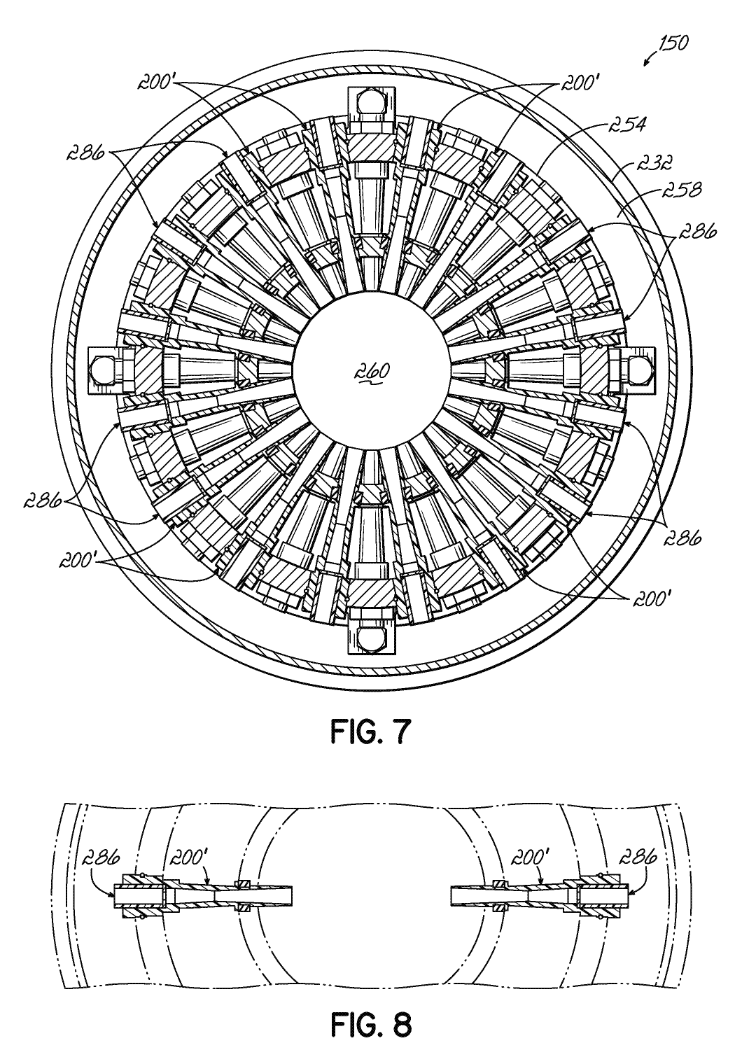

As seen in FIGS. 4 and 7, it is contemplated that hundreds, perhaps even a thousand or more of cyclonettes, as depicted at 200′, will be arrayed in a single housing 230. In one embodiment, each cyclonette 200′, as shown in FIG. 8, is disposed opposite another, resulting in direct impingement of the flow from one cyclonette upon the opposite flow from an opposing cyclonette.

Conventional utilization of a cyclonette and vortex finder insert as shown in U.S. Pat. No. 5,388,708, for example, would result in flow, with reference to FIG. 5, into the intermediate manifold 252 and thence, with reference to FIG. 4, into the intermediate chamber 262. From there the flow would pass into the passageway 226 as seen in FIG. 9 of the drawings, and then spiral around the surface of the throat 212 and thereafter, around the surface of the tapered flow channel 214 to the right as seen in FIG. 9 of the drawing. This would set up a counter flow to the left as seen in FIG. 9 and out the vortex finder 202 of the fines fraction of the suspension while the heavier fractions of the suspension passed on out the narrower flow channel 216 of the cyclonette.

In contrast, in accordance with the liquid treatment apparatus 150 of the present invention, the feed flow in manifold 248, as shown in FIG. 5, is just the opposite of conventional operation. That is, instead of accepting the fines in an outward flow, the manifold 248 is in fact the feed manifold for the system, delivering the liquid medium processing stream, to be treated to the upstream or left-hand end of the vortex finder, as shown in FIG. 9, from whence the flow is ejected in an axial jet out the extension 224 of the vortex finder and into the tapering flow channel 214. This action results in the generation of shear zones that create a myriad of tiny bubbles, each of which, upon implosion, create highly localized areas of extreme pressures and temperatures.

This in turn results in dissolution of the water molecules into, inter alia, aggressive hydroxyl radicals. While in its most straightforward form the passageway 226 in the upstream end of the cyclonette will not be utilized, in a modification of the basic form of the invention, a supply of the liquid medium processing stream may be fed via the intermediate manifold 252 and the intermediate chamber 262 into the passageways 226 to provide an additional flow and hence an intensifying of the shear zone to enhance the formation of the tiny bubbles as liquid flows through the tapering flow channel 214 of the cyclonette 200.

Depending upon the desired effect, the passageway 226 may be disposed tangentially with respect to the throat 212, radially, or even substantially axially. It should also be noted that, in addition to utilizing the passageway 226 for the supplemental flow of the liquid being treated, different fluids, gaseous or liquid, optionally could be injected through the passageway 226 to alter the physical or chemical character of the liquid medium processing stream being treated. For example, a pH-adjusting fluid, if desired, could be supplied through the passageway 226.

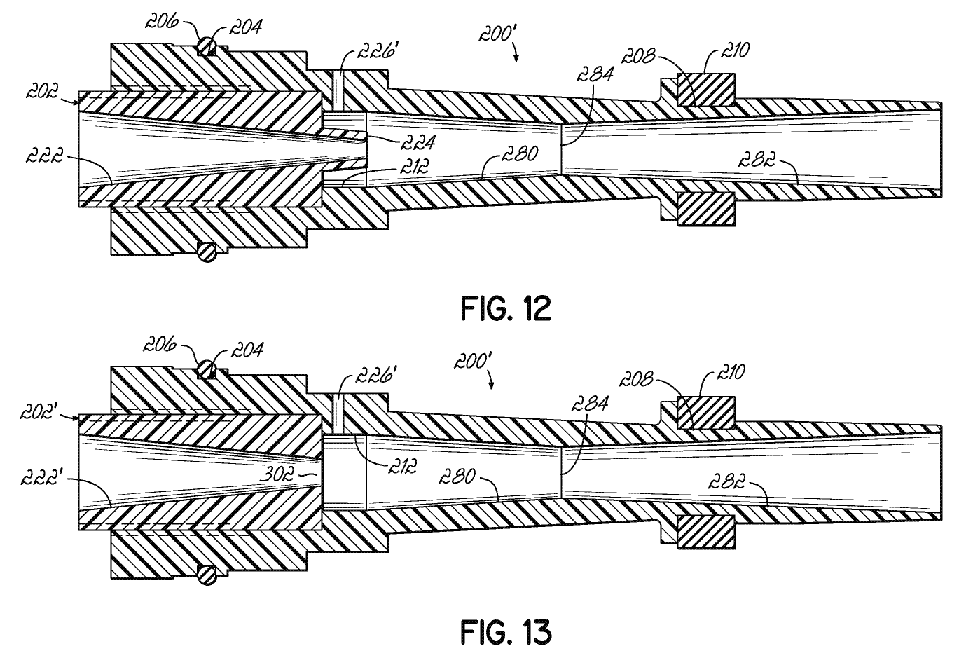

FIG. 12 of the drawings shows cyclonette 200′, similar to that of FIG. 9, but with flow channels 214 and 216 replaced by flow channels 280 and 282. The reduced diameter at point 284 results in an increase in velocity and a corresponding reduction in static pressure. The pressure within the chamber is directly related to the velocity head at this point. The outwardly tapering flow channel 282 results in a gradual decrease in fluid velocity, permitting efficient conversion of velocity head into static head as the fluid moves toward the discharge zone.

As seen in FIG. 13, cyclonette 200′ is provided with the vortex finder 202 of FIGS. 9 and 12 being replaced by vortex finder 202′ in which the extension 224 (FIG. 9) protruding into the throat portion 212 is eliminated. As a result, the immediate transition from the downstream end of the modified vortex finder 202′ into the larger diameter throat portion 212 provides an additional shear zone for the generation of the desirable fine bubbles.

In yet another modification of the liquid treatment apparatus 150 of the present invention, as shown in FIG. 10, cyclonette 200′ is combined with insert 286 having a straight sided internal bore 288 and external threads 289, which are complementary to internal threads 218′ in the modified cyclonette 200′. The insert 286 captures and holds in place within the cyclonette 200′ a washer-shaped orifice plate 300 having a central orifice 302. This embodiment results in the formation of multiple tiny bubbles, as the liquid being treated must first constrict from the larger diameter of the insert flow passage 288 to the restricted orifice 282 and then expand again into the throat 212 of the cyclonette 200′. In this embodiment, as in those of FIGS. 12 and 13, the passageway 226′ may be used for the addition of a flow of the liquid medium processing stream or a chemical or physical modifying substance in either a tangential, radial or substantially axial direction into the throat 212 of the cyclonette 200 or 200′.

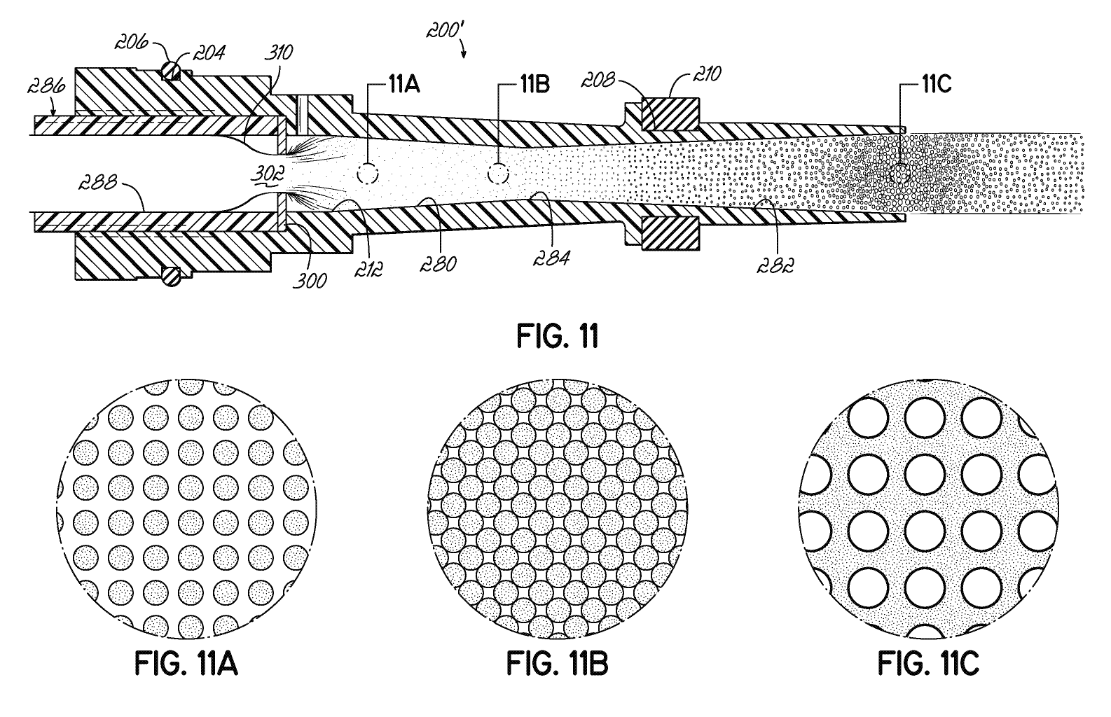

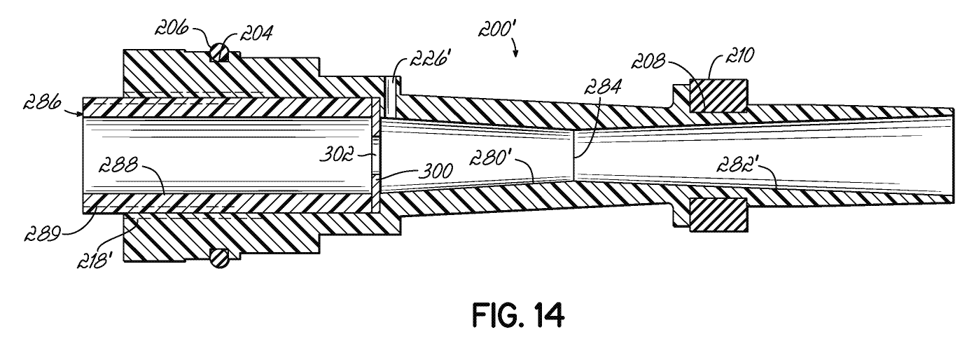

In some cases, it may be found desirable to eliminate the throat 212, as shown in FIG. 14 of the drawings, and convey the flow through the orifice 302 directly into an inwardly tapered flow channel 280′ and then outwardly into the outwardly tapering flow channel 282′. In this embodiment, as in the embodiments of FIGS. 10 and 11, the orifice plate 300 is held in place in the cyclonette 200′ by the insert 286, which permits orifice plate 300 to be easily replaced for wear or the like.

Turning now to FIGS. 11, 11A, 11B and 11C, it will be seen that liquid medium processing stream 310 that is delivered during alcohol production to the upstream end of a modified cyclonette 200′, via the outer manifold 248 and outer annular chamber 258, passes through an insert 286 and thence through the orifice 302 of the orifice plate 300 and into the throat portion 212. This creates an intense shear zone, resulting in a myriad of fine bubbles and droplets, some of which are dispersed at point 11A in the flow channel 280 as depicted diagrammatically in FIG. 11A. As the flow proceeds downstream through the ever-narrowing flow channel, the droplets move closer together and entrain pockets of vapor. Some of the kinetic energy of the liquid medium processing stream 310 is utilized to accelerate and compress the pockets of vapor into bubbles until downstream flow channel 282 is reached. Beyond point 11B, as the fluid moves to a zone of expanding diameter, the bubbles tend to expand. Lastly, at point 11C, the bubbles have assumed a size and configuration as shown in FIG. 11C of the drawings.

Thus, it will be seen that the liquid treatment apparatus 150 utilizes a vacuum chamber maintained within the individual cyclonettes 200, 200′ by immersing their discharge ends in the liquid medium processing stream 310 being treated and directing a high velocity jet of that liquid 310 to pass through a volume of vapor to increase bubble formation once vacuum is achieved. When these bubbles collapse, localized temperatures of 5,000 degrees Kelvin or more, and pressures of more than one thousand atmospheres can be achieved. This can produce profound physical and chemical reactions. The collapse of bubbles under these conditions also generates shock waves that propagate within the fluid media. The energy transferred by these shock waves can also result in physical and chemical changes to materials within the fluid.

From the above, it will be apparent that the liquid treatment apparatus 150 provides an efficient method of harnessing the water molecule dissolution powers of hydraulic cavitation with the consequent release of aggressive hydroxyl radicals and highly effective liquid treatment. Additionally, the liquid treatment apparatus 150 utilizes conventional hydrocyclones and modifications thereof by operating them in a manner completely contrary to their intended purpose. To that end, while the liquid treatment apparatus 150 is described herein as being equipped with cyclonettes 200, 200′ for generating hydraulic cavitation, it should be understood that other liquid treatment apparatuses may be utilized for subjecting the liquid medium processing stream to shear under vacuum to generate hydraulic cavitation.

The various embodiments of the present invention provide for the use of the liquid treatment apparatus 150 at various points of an alcohol production process to effect desired changes to the fluid medium and/or components flowing in the medium. Use of the liquid treatment apparatus 150 in this manner has multiple benefits, including, but not limited to, increase in efficiency of alcohol production, production of marketable by-products, and the like, as will be described in more detail herein.

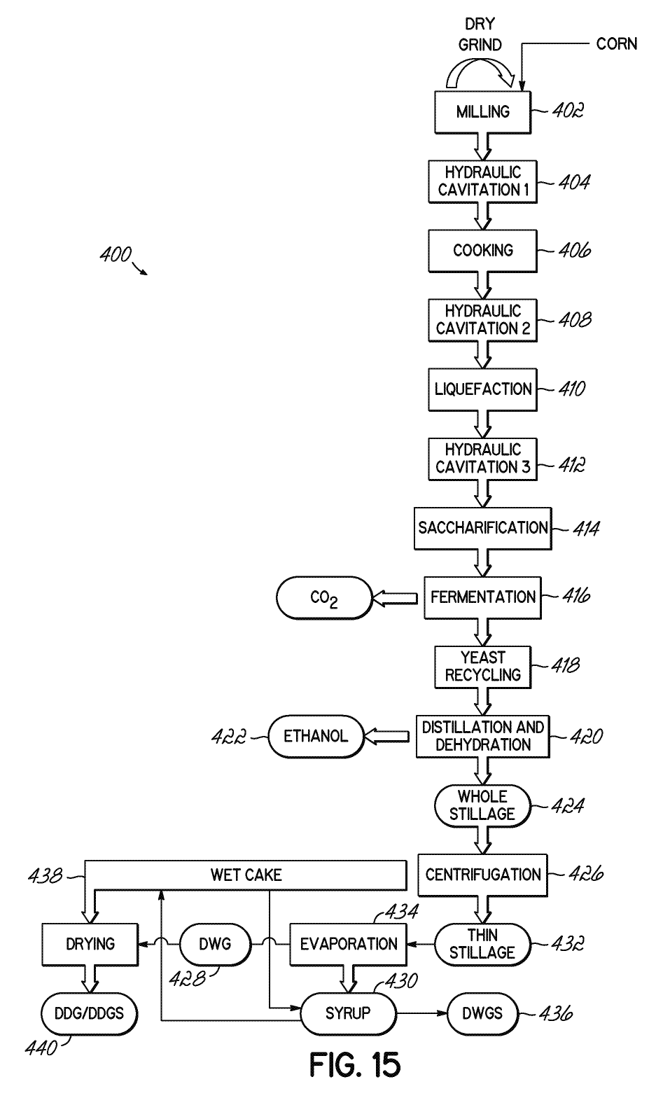

Accordingly, liquid treatment apparatus 150, as generally identified by “hydraulic cavitation” in the diagrams in FIGS. 15-18, may be inserted before, after, or between one or more processing steps of an alcohol production process to receive the moving fluid medium, i.e., the liquid medium processing stream 310 (FIG. 11), which can contain large particulates. The liquid treatment apparatus 150 can be installed along with the various pieces of process machinery in the alcohol production process by means known to those having ordinary skill in the art. Specific placement of the liquid treatment apparatus 150 in the stream will vary depending upon the application. In some embodiments, multiple liquid treatment apparatuses 150 can be placed in parallel or in series in the process to receive the moving fluid medium. The hydraulic cavitation generated by the liquid treatment apparatus 150 interacts directly with the moving fluid medium, which can cause large particulates to be broken down into small particulates.

The benefits of hydraulic cavitation occurring in the alcohol production stream are significant. For example, hydraulic cavitation of the moving fluid medium can allow for destructuring, disaggregation, and disassociation of starch granules from other grain components such as protein and fiber that may inhibit the conversion of starch to glucose and ethanol. The cavitational forces provided by the liquid treatment apparatus 150 may loosen, shake off and/or strip away starch granules from protein bodies, protein matrices, and fiber (fine or coarse), as well as disassociate tightly packed granules and tightly packed amyloplasts which contain starch granules. It is important to note, however, that overprocessing of the components, e.g., overprocessing of starch prior to fermentation, is not desirable. Specifically, if the applied cavitation is too aggressive in terms of intensity, frequency, and/or duration, it may be possible to cause some damage to the components being treated. For example, care must be taken not to degrade desirable proteins, enzymes, or damage the yeast. Additionally, care must also be taken to not shear the starch to the point that it is all converted into sugar too quickly, which could also inhibit or kill the yeast. Therefore, more intense cavitation is limited to specific uses that may be considered less sensitive to this type of concern. This includes applications that do not require the enzymes or yeast to be present.

Hydraulic cavitation of the fluid helps to enable the other changes taking place with the particulates. Specifically, disassociation of water molecules into hydrogen ions [H+] and hydroxyl groups [OH−] creates “free radicals,” i.e., miniature “chemical reactors,” which operate at a localized level to enable some of the benefits described herein, particularly those requiring greater “destruction” of the components, e.g., denaturing or degradation of transgenic proteins and transgenic nucleic acids of genetically modified feedstocks, rendering of bacteria and/or fungus and/or yeast as nonviable, and the like.

The required level of hydraulic cavitation, which may be varied by the design of the cyclonette, for example, can be identified by measuring the conversion rates, e.g., speed of liquefaction or speed of fermentation, and intermediate or final product yields of the particular step of interest, while varying the type of cyclonette used. Additionally, some of the benefits of creating cavitational forces at various locations in an alcohol production process include, but are not limited to, increased alcohol fermentation, i.e., faster fermentations and/or higher alcohol yields, decreased chemical and biological additives, reduction of energy costs (e.g., key processes such as cooking are completed at lower temperatures), denaturation or degradation of transgenic proteins and transgenic nucleic acids of genetically modified feedstocks and rendering nonviable bacteria and/or fungi and/or yeast contaminants. The benefit or benefits obtained will vary depending on whether the alcohol production process is a dry grind process, a modified dry grind process, or a wet mill process. Achieving a particular benefit, within a particular type of process, however, is dependent on many factors, including the location or locations in the process at which the liquid treatment apparatus 150 is utilized, the intensity and frequency of the hydraulic cavitation, alcohol production process variables, and the like.

In one embodiment, hydraulic cavitation is utilized only once during the alcohol production process in just one location of the liquid medium processing stream, with one liquid treatment apparatus 150. In other embodiments, hydraulic cavitation is utilized in more than one location using multiple liquid treatment apparatuses 150 to increase and/or vary the benefits obtained. The liquid medium processing stream can include, but is not limited to, heavy steep water, uncooked slurry, cooked mash, liquefied mash, and (for dry grind processes) whole stillage, thin stillage and wet cake.

FIGS. 15-18 are flow diagrams showing methods for producing ethanol from corn by including the liquid treatment apparatus 150, which generates hydraulic cavitation, at one or more locations in a dry grind ethanol production process 400, a modified dry grind ethanol production process 500, and a wet mill ethanol production process 600, respectively, although the invention is not so limited. Again, hydraulic cavitation applied as described herein is also useful in other grain-based ethanol production facilities which rely on various other grains including wheat, barley, sorghum, oats, rice and the like. Additionally, hydraulic cavitation is also useful for grain-based production facilities that produce alcohols other than ethanol. Such alcohols include, but are not limited to, industrial alcohols such as methanol, isopropanol, butanol, and so forth, further including propane diol, which can be used to make bioplastics. It is also likely that hydraulic cavitation would be useful in grain-based production facilities that produce various organic acids, such as lactic acids. Most likely such production facilities which produce alcohols other than ethanol and/or organic acids are wet mill processes which utilize an alternative fermentation process although it may also be possible to use a dry grind or modified dry grind process to produce these products.

FIG. 15 is a diagram showing methods of ethanol production using the liquid treatment apparatus 150 to generate hydraulic cavitation at one or more locations in the dry grind ethanol production process 400. The process begins as described above for FIG. 1 with corn being milled in a milling step 402. A first hydraulic cavitation step (hydraulic cavitation 1) 403 can occur just after the milling step 402, i.e., prior to the cooking step 406, which can be a jet or non-jet cooking step. Application of hydraulic cavitation, via liquid treatment apparatus 150, to the uncooked slurry of the liquid medium processing stream at this point can cause protein and fiber to be stripped from the starch, thus enhancing gelatinization. Specifically, the resulting cavitation can make the starch granules more accessible and available to water molecules to increase the rate of gelatinization of the entire population of starch granules. This results in shorter holding times for the gelatinization process, which provides a cost reduction benefit by reducing the input energy to maintain the desired temperature of the solution as well as a net increase of production capacity via higher plant throughput. Enhancement in starch gelatinization also helps to speed up liquefaction.

Additionally or alternatively, a second hydraulic cavitation step (hydraulic cavitation 2) 408 can occur just after the cooking step 406. Such hydraulic cavitation again can cause protein and fiber to be stripped from the starch, thus enhancing liquefaction. In some embodiments, the liquefaction holding time and/or required alpha-amylase amount to achieve liquefaction can be reduced when hydraulic cavitation is used around the cooking step 406.

It is important to note that it is undesirable to overprocess the starch, particularly prior to fermentation. Testing can determine the most beneficial location for hydraulic cavitation around the cooking step. Therefore, the use of hydraulic cavitation before and/or after cooking will vary depending on the specific process, benefits desired, and so forth. It is also possible that applying hydraulic cavitation to the uncooked slurry may allow the cooking step 406 to be a non-jet cooking step versus a jet cooking step. In other embodiments, hydraulic cavitation of the slurry around the cooking step 406 can allow for lower jet cooking temperatures and/or shorter cooking times while still achieving optimal gelatinization of the starch. At the very least, hydraulic cavitation in this area should reduce energy costs related to the cooking step, such as the costs associated with providing steam.

Additionally or alternatively, a third hydraulic cavitation step (hydraulic cavitation 3) 412 can occur after the liquefaction step 410. Hydraulic cavitation, via the liquid treatment apparatus 150, of exiting liquefied mash at this point in the process can cause disruption of starch and maltodextrins, resulting in enhanced saccharification. Hydraulic cavitation at this point also can reduce the amount of gluco-amylase required to achieve optimal saccharification and will also reduce the holding time for the subsequent saccharification step 414 and fermentation step 416. In some embodiments, the saccharification step 414 and fermentation step 416 occur simultaneously as described above in FIG. 1, i.e., SSF. Hydraulic cavitation is also not likely to be used immediately after the saccharification step 414, although it could be in embodiments in which the saccharification step 414 and fermentation step 416 are performed separately. To that end, it should be understood by one having ordinary skill in the art that the liquid treatment apparatus 150 for generating hydraulic cavitation may be provided at other various points along the dry grind ethanol production process.

After the fermentation step 416 there is the optional yeast recycling step 418 and distillation and dehydration step 420 which produces ethanol 422 and whole stillage 424 as discussed above in FIG. 1. This is followed by centrifugation step 426. The DWG 428 (produced along with syrup 430 after the thin stillage 432 goes through the evaporation step 434), DWGS 436 (centrifuged wet cake 438 and syrup 430) and/or DDG/DDGS 440 (dried wet cake 438) produced downstream, can provide animal feeds (including pet foods) having proteins (known in the art) which are not normally bio-available to the digestive system of most animals, including, but not limited to, swine, poultry, beef and dairy cattle, and the like, further including domesticated animals.

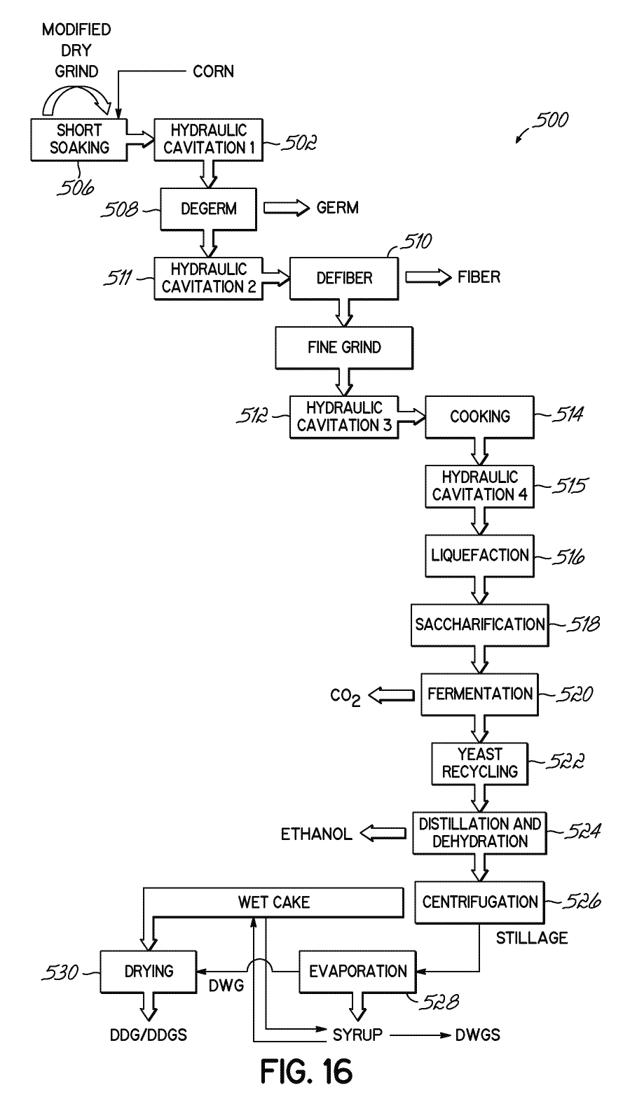

FIG. 16 is a diagram showing methods of ethanol production using the liquid treatment apparatus 150 to generate hydraulic cavitation at one or more locations in the modified dry grind ethanol production process 500. The application of hydraulic cavitation to corn fiber derived from any modified dry grind ethanol process can improve the extraction efficiency and yield of fiber oils, fiber phytosterols, fiber gums, fiber carotenoids, and fiber tocopherols, and any other nutraceuticals and chemicals extracted from corn fiber. The application of hydraulic cavitation to any existing modified dry grind ethanol process can also improve the efficiencies, yields, and quality of existing corn defiber and degerm technologies in which corn germ and coarse fiber (pericarp), are removed and separated from the remaining corn components.

In this embodiment, a first hydraulic cavitation step (hydraulic cavitation 1) 502 can occur just after the short soaking 506, i.e., prior to the degerm step 508. Hydraulic cavitation, via liquid treatment apparatus 150, of the uncooked slurry of the liquid medium processing stream at this point can cause germ to pop out more efficiently, possibly reducing the amount of grinding needed in subsequent steps. Hydraulic cavitation at this point may also reduce the amount of degerming required in the degerm step 508. In one embodiment, use of the first hydraulic cavitation step 502 removes and separates corn germ from the remaining corn grain components, thus eliminating the need for the degerm step 508 altogether. The first hydraulic cavitation step 502 may also enable both corn germ and coarse fiber (pericarp) to be simultaneously stripped away from the endosperm, possibly reducing the amount of grinding required downstream. Hydraulic cavitation at this point may also eliminate the need for both the degerm step 508 and the defiber step 510.

Additionally or alternatively, a second hydraulic cavitation step (hydraulic cavitation 2) 511 can occur between the degerm step 508 and defiber step 510. Use of hydraulic cavitation, via liquid treatment apparatus 150, to the uncooked slurry at this point, helps to remove the coarse fiber (pericarp), from the remaining corn grain components, thus reducing the amount of fiber that needs to be separated in the defiber step 510. Hydraulic cavitation at this point may also eliminate the need for the defiber step 510 altogether.

Additionally or alternatively, a third hydraulic cavitation step (hydraulic cavitation 3) 512 can occur before the cooking step 514, which again can be a jet cooking or non-jet cooking process. Additionally or alternatively, a fourth hydraulic cavitation step (hydraulic cavitation 4) 515 can be provided just after the cooking step 514. Hydraulic cavitation, via liquid treatment apparatus 150, of the uncooked slurry or the resulting cooked mash of the liquid medium processing stream at these points, respectively, in the process again can cause protein and fine fiber to be stripped from the starch, thus enhancing liquefaction. Again, in some embodiments, liquefaction holding time and/or required alpha-amylase amount to achieve liquefaction is reduced.

As noted above in reference to FIG. 15, hydraulic cavitation is also not likely to be used immediately after the saccharification step 518, although it could be in embodiments in which the saccharification step 518 and fermentation step 520 are performed separately. To that end, it should be understood by one having ordinary skill in the art that the liquid treatment apparatus 150 for generating hydraulic cavitation may be provided at other various points along the dry grind ethanol production process.

After yeast recycling step 522, FIG. 16 further shows a distillation and dehydration step 524 which produces ethanol as described in FIG. 1. The subsequent centrifugation step 526 centrifuges the residuals produced with the distillation and dehydration step 524, as described in FIG. 1, to produce stillage and wet cake as shown in FIG. 16. Additionally, although it is also possible to apply hydraulic cavitation to the stillage since the fiber, germ, and other grain insoluble components have been removed at this stage of the process, this stillage has very little insoluble solids present and any benefits achieved may be limited. FIG. 16 further shows the stillage going through an evaporation step 528 to produce syrup and DWG. The syrup can be mixed with the wet cake to produce DWGS as shown in FIG. 16 and described in FIG. 1. Alternatively, as shown in FIG. 16 (and described in FIG. 1), the wet cake and syrup may be dried in a drying step 630 to produce DDG/DDGS.

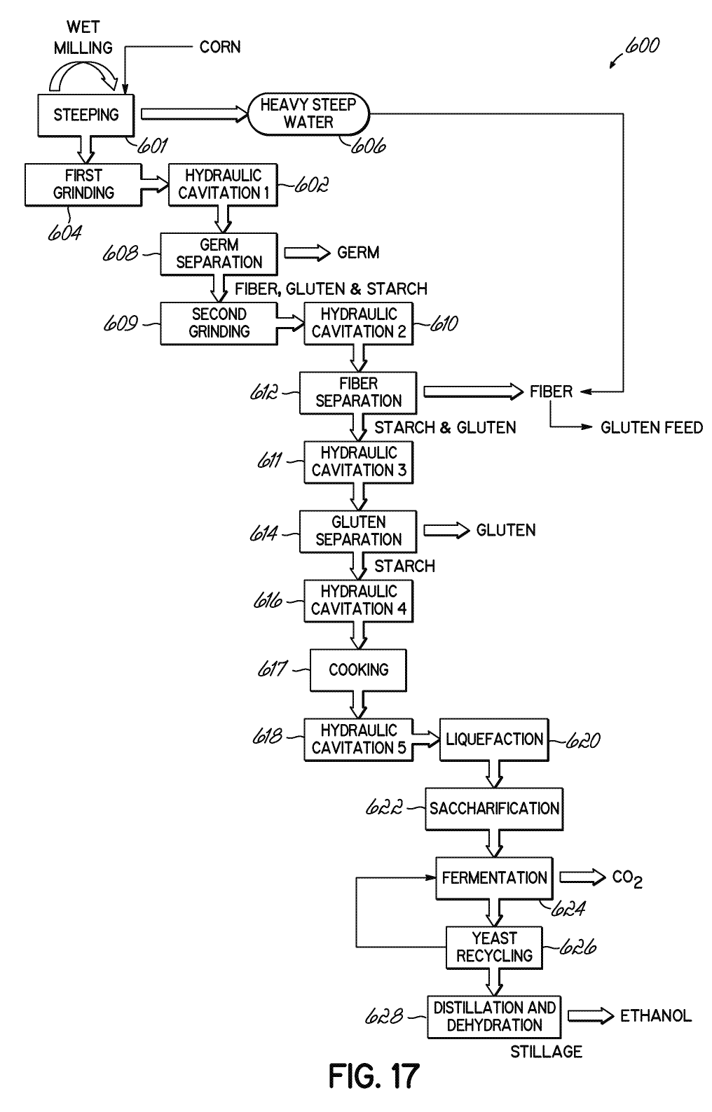

FIG. 17 is a diagram showing methods of ethanol production using the liquid treatment apparatus 150 to generate hydraulic cavitation at one or more locations in a wet mill ethanol production process 600. Generally speaking, use of hydraulic cavitation in a wet mill process produces cavitational forces that can loosen, shake off, or strip away starch granules from protein bodies, protein matrices, and fiber (fine or coarse), as well as disassociate tightly packed granules and tightly packed amyloplasts which contain starch granules. The net effect is that hydraulic cavitation can generate higher yields of starch granules in the final starch stream, and less residual starch in the fiber stream and gluten (protein) stream.

In this embodiment, a first hydraulic cavitation step (hydraulic cavitation 1) 602 can occur just after the first grinding step 604, which is after the steeping step 601. Hydraulic cavitation of the uncooked slurry of the liquid medium processing stream, via liquid treatment apparatus 150, at this point in the process can result in enhanced separation of germ from the corn kernel in step 608, as well as enhanced separation of fiber from starch and gluten in the fiber separating step 612 downstream. Although not shown, hydraulic cavitation can be applied to the steeping water used in the steeping step 601 as well as the heavy steep water 606, i.e., concentrated steep water (syrup) produced as a result of the steeping step 601. Hydraulic cavitation, via the liquid treatment apparatus 150, at those points can cause degradation or denaturation of transgenic nucleic acids and protein.

Additionally or alternatively, a second hydraulic cavitation step (hydraulic cavitation 2) 610 can occur just after the second grinding step 609, such cavitation being applied to uncooked slurry of the liquid medium processing stream. Additionally or alternatively, a third hydraulic cavitation step (hydraulic cavitation 3) 611 can occur just after the fiber separation step 612. At this point, the hydraulic cavitation is applied to the aqueous stream of starch and gluten prior to the gluten being separated from the starch in the gluten separation step 614 via any suitable method. Hydraulic cavitation at this point in the process can also result in enhanced separation of starch and gluten.

Additionally or alternatively, a fourth hydraulic cavitation step (hydraulic cavitation 4) 616 can occur before the cooking step 617, i.e., just after the gluten separation step 614. Again, the cooking step 617 can be a jet cooking or non-jet cooking process. Hydraulic cavitation, via liquid treatment apparatus 150, at this point in the process can enhance starch gelatinization and liquefaction.

Additionally or alternatively, a fifth hydraulic cavitation step (hydraulic cavitation 5) 618 can occur just after the cooking step 617. Hydraulic cavitation of the resulting cooked mash, via liquid treatment apparatus 150, at this point in the process again can cause protein and fiber to be stripped from the starch, thus enhancing liquefaction. Again, in some embodiments, liquefaction holding time and/or required alpha-amylase amount to achieve liquefaction is reduced.

As noted above in reference to FIG. 15 hydraulic cavitation is also not likely to be used immediately after the liquefaction step 620 or the saccharification step 622, although it could be in embodiments in which the saccharification step 622 and fermentation step 624 are performed separately. To that end, it should be understood by one having ordinary skill in the art that the liquid treatment apparatus 150 for generating hydraulic cavitation may be provided at other various points along the dry grind ethanol production process. Next, the fermentation step 624 is followed by yeast recycling step 626 as shown in FIG. 17 and discussed in FIG. 3. Additionally, although it is also possible to apply hydraulic cavitation to the stillage (produced after the distillation and dehydration step 628 as shown in FIG. 17), since the fiber and germ have been removed at this stage of the process, the stillage has very little or no insoluble solids present and any benefits achieved may be limited.

In one embodiment of the present invention, hydraulic cavitation that is applied, via liquid treatment apparatus 150, to the liquid medium processing stream after whole kernel milling and before and/or after cooking of starch in the dry grind, modified dry grind, or wet mill ethanol process 400, 500, or 600 can cause stripping away of cell macromolecules, such as protein and fiber from the surface of starch granules. That hydraulic cavitation can also cause the opening or breaking of gelatinized starch granules, all of which can make starch granules more accessible and available to enzymes during liquefaction and saccharification in dry grind, modified dry grind and wet mill ethanol processing. Similarly, hydraulic cavitation that is applied, via liquid treatment apparatus 150, to the liquid medium processing stream after cooking in a dry grind, modified dry grind or wet mill ethanol process 400, 500, or 600 according to the present invention can cause gelatinized starch granules to open and/or partially disintegrate, thus making them more accessible. The overall enabling impact is that hydraulic cavitation generated by the liquid treatment apparatus 150 creates greater levels of fermentable starch (in a dry grind process), or extracted starch (in a wet mill process), thus increasing the yield of ethanol as a function of the total starch input. Another consequence is that DDGS (a co-product of the dry grind process) will contain lower levels of residual starch as it will have been converted to ethanol. It is more desirable to have the lowest possible quantities of starch in DDGS because the starch value is realized in ethanol having a greater commercial value than DDGS. As a result, the DDGS will be higher in protein which enhances the value of DDGS as an animal feed.

In one embodiment, hydraulic cavitation that is applied, via liquid treatment apparatus 150, to the liquid medium processing stream before or after liquefaction in a dry grind, modified dry grind or wet mill ethanol process 400, 500, or 600 according to the present invention can allow hydrolyzation or depolymerization of long polymeric macromolecules such as starch, protein, and at very high power levels, nucleic acids. By breaking down the various macromolecules, hydraulic cavitation can increase the rate of liquefaction and saccharification of the starch by making the components more accessible to alpha-amylase and gluco-amylase, the normal active enzymes used in liquefaction and saccharification.

In one embodiment, hydraulic cavitation that is applied, via liquid treatment apparatus 150, to the liquid medium processing stream of the commercial ethanol process at one or more points prior to (upstream to) fermentation, kills contaminating microorganisms through cell lysis and/or cell damage, thereby reducing the possibility of microbial contamination during fermentation. Contaminating microorganisms include bacteria, fungi (mold), and yeasts. The application of hydraulic cavitation prior to fermentation also reduces or eliminates the requirement to add exogenous protease enzymes which hydrolyze protein to make starch more accessible for hydrolysis and fermentation.

In one embodiment, hydraulic cavitation generated by the liquid treatment apparatus 150, when applied to the liquid medium processing stream of the commercial ethanol process at one or more points prior to (upstream of) fermentation or subsequent to (down stream from) fermentation, can degrade, depolymerize (hydrolyze), or denature mycotoxins produced by molds which are present in the incoming corn feedstock. By detoxifyinig mycotoxins through degradation or depolymerization, mycotoxin levels can be drastically reduced or eliminated in DWGS and DDGS, thus allowing these components to readily achieve safe toxicity levels for animal feed purposes. Therefore, use of hydraulic cavitation as described herein will allow ethanol plant grain deliveries, which normally would be rejected due to unacceptable fungal and mycotoxin loads, to be accepted for ethanol processing.

When applied to the liquid medium processing stream of any of the processes listed above, hydraulic cavitation generated by the liquid treatment apparatus 150 can increase ethanol plant throughput, reduce energy and enzyme input costs, increase ethanol yields, and reduce residual starch in DWGS or DDGS. When applied to any of the processes described herein at one or more points, hydraulic cavitation generated by the liquid treatment apparatus 150 can increase ethanol plant throughput, reduce energy and enzyme input costs, increase ethanol yields and reduces residual starch in DWGS or DDGS.

In one embodiment, hydraulic cavitation generated by the liquid treatment apparatus 150, when applied to the liquid medium processing stream of any commercial dry grind, modified dry grind, or wet mill ethanol process, in which the feedstock consists of genetically modified corn, at one or more points in the process can degrade, depolymerize (hydrolyze), or denature transgenic deoxyribonucleic acid (DNA), transgenic ribonucleic acid (RNA), and transgenic proteins derived from genetically-modified corn. The degradation, depolymerization, or denaturation of transgenic DNA, RNA, and protein can be adequately severe as to render transgenic DNA, RNA, and protein as undetectable by standard methods of analysis of primary products and co-products from any commercial wet mill or dry grind ethanol process. As a result, hydraulic cavitation can render any primary product and co-product acceptable for export to countries that have not yet approved import of food and feed products derived from genetically modified corn. Primary products and co-products include but are not limited to ethanol, DDGS and DWGS from the dry mill (dry grind) ethanol process, as well as starch, germ, gluten feed, and gluten meal from the wet mill ethanol process. Standard methods of analysis for transgenic DNA, RNA, and protein, include but are not limited to polymerase chain reaction (PCR) detection methods, Southern blot methods, Northern blot methods and dipstick hybridization methods, as well as immunological detection methods such as Western blot methods and Enzyme-Linked Immuno-Sorbent Assay (ELISA) methods, as is known in the art.

In one embodiment, complex proteins (i.e., proteins not normally bio-available to the digestive systems of many animals, i.e., proteins not susceptible to hydrolysis to amino acids by proteolytic enzymes) present in whole stillage are affected by application of hydraulic cavitation, producing novel animal feeds having proteins which are less complex and therefore more bio-available to the digestive systems of many animals. The proteins are affected in any number of ways with hydraulic cavitation, including but not limited to, being shaken loose or stripped away from starch granules or fiber, thus making the protein more available for hydrolysis by digestive (proteolytic) enzymes. Proteins associated as complexes and protein matrices are also being disrupted and disassociated to make them more available for hydrolysis by digestive (proteolytic) enzymes. Proteins are also being mechanically hydrolyzed by cavitational forces into short chain peptides, which are more readily further hydrolyzed by digestive (proteolytic) enzymes.

In one embodiment, hydraulic cavitation generated via the liquid treatment apparatus 150 is used for the improvement in process efficiency, product yield, speed, or product quality of any processing step throughout the commercial dry grind ethanol process, or for any type and design of modified dry grind ethanol process or wet mill process. This includes, but is not limited to the application of hydraulic cavitation via liquid treatment apparatus 150 to improve the yield of ethanol production, or the rate (speed) of ethanol production, or the combination of the yield of ethanol and rate (speed) of ethanol production, and the application of hydraulic cavitation to reduce or eliminate processing inputs such as quantity of enzymes, quantity of heat and energy, and quantity of chemicals.

Application of hydraulic cavitation, via liquid treatment apparatus 150, to one or more of the various processing streams in a dry grind, wet mill or modified dry grind ethanol process 400, 500, or 600 can be accomplished with relatively minor retrofitting of existing equipment. Essentially, the liquid treatment apparatus 150 can easily be interfaced with or integrated into existing processing steps and technologies, thus allowing ethanol producers to overcome technological hurdles, inefficiencies, and poor yields in an easy and cost efficient manner without the need to undergo costly and time-consuming re-tooling of their facilities. Additionally, the liquid treatment apparatus 150 may potentially be used at one or more points of other alcohol production processes to provide enhancements and benefits as described herein.

Although specific embodiments have been illustrated and described herein, it will be appreciated by those of ordinary skill in the art that any arrangement that is calculated to achieve the same purpose may be substituted for the specific embodiment shown. For example, although the various systems and methods described herein have focused on corn, virtually any type of grain, including, but not limited to, wheat, barley, sorghum, rye, rice, oats and the like, can be used. This application is intended to cover any adaptations or variations of the present subject matter. In addition, simple modifications to the liquid treatment apparatus 150 may be required, for example, to reduce blockage issues associated with pumping liquid medium processing streams containing fibers. In that case, a larger apparatus design is one option. Also, while the liquid treatment apparatus 150 is described herein as primarily being equipped with cyclonettes 200, 200′ for generating hydraulic cavitation, other liquid treatment apparatuses may be used for subjecting the liquid medium processing stream to shear under vacuum to generate hydraulic cavitation. Therefore, it is manifestly intended that embodiments of this invention be limited only by the claims and the equivalents thereof.

What is claimed is:

1. A method for alcohol production comprising:
at one or more points along an alcohol production process, feeding a grain-based liquid medium processing stream of the alcohol production process through a liquid treatment apparatus configured to generate hydraulic cavitation; and
generating hydraulic cavitation within the liquid treatment apparatus by subjecting the grain-based liquid medium processing stream to shear under vacuum within the liquid treatment apparatus.

2. The method of claim 1 wherein the grain-based liquid medium processing stream is selected from the group consisting of corn, rye, sorghum, wheat, barley, oats, and rice.

3. The method of claim 1 wherein the alcohol production process is a dry grind, modified dry grind, or wet mill ethanol production process.

4. The method of claim 3 wherein the dry grind, modified dry grind, or wet mill starch-to-ethanol production process includes a fermentation step, and wherein hydraulic cavitation is generated by subjecting the grain-based liquid medium processing stream to shear under vacuum within the liquid treatment apparatus prior to the fermentation step at one or more locations.

5. The method of claim 1 wherein the alcohol production process is a starch-to-alcohol production process.

6. The method of claim 1 wherein the liquid treatment apparatus comprises:
a plurality of cyclonettes, each cyclonette including an upstream and a downstream end and an internal, unidirectional flow channel extending through said cyclonettes from said upstream end to said downstream end;
a feed channel communicating with said upstream ends of said cyclonettes and feeding said grain-based liquid medium processing stream to said upstream ends thereof; and
an outwardly-flowing channel communicating with and immersing said downstream ends of said cyclonettes in said grain-based liquid medium processing stream and conveying said grain-based liquid medium processing stream away from said downstream ends of said cyclonettes.

7. The method of claim 6 wherein the flow channel includes a first portion tapering inwardly in a downstream direction.

8. The method of claim 6 wherein the liquid treatment apparatus further comprises a throat portion of substantially constant internal diameter positioned upstream of said upstream end of said unidirectional flow channel.

9. The method of claim 6 wherein the liquid treatment apparatus further comprises a vortex finder received in each of said cyclonettes adjacent said upstream end thereof.

10. The method of claim 6 wherein the liquid treatment apparatus further comprises:
an orifice plate having an orifice defined therethrough positioned in each of said cyclonettes adjacent said upstream end of said unidirectional flow channel; and
said orifice having a diameter smaller than that of said flow channel at said upstream end thereof.

11. The method of claim 6 wherein the liquid treatment apparatus further comprises:
first and second, concentric, cylindrical casings defining therebetween said feed channel, and
said plurality of cyclonettes being mounted in said second cylindrical casing.

12. The method of claim 6 wherein the liquid treatment apparatus further comprises a passageway extending through a wall of each of said cyclonettes.

13. A method for alcohol production comprising:
applying hydraulic cavitation to a grain-based liquid medium processing stream at one or more locations in an alcohol production process, the hydraulic cavitation being applied by a liquid treatment apparatus wherein the liquid treatment apparatus subjects the grain-based liquid medium processing stream to shear under vacuum within the liquid treatment apparatus.

14. The method of claim 13 wherein in the grain-based liquid medium processing stream is selected from the group consisting of corn, rye, sorghum, wheat, barley, oats, and rice.

15. The method of claim 13 wherein the alcohol production process is a dry grind, modified dry grind, or wet mill ethanol production process.

16. The method of claim 15 wherein the dry grind, modified dry grind, or wet mill starch-to-ethanol production process includes a fermentation step, and wherein hydraulic cavitation is applied to the liquid medium processing stream prior to the fermentation step at one or more locations.

17. The method of claim 13 wherein the alcohol production process is a starch-to-alcohol production process.

18. The method of claim 13 wherein the liquid treatment apparatus includes a feed channel, an outwardly-flowing channel, and a plurality of cyclonettes that generate hydraulic cavitation, each cyclonette including an upstream and a downstream end and an internal, unidirectional flow channel extending through said cyclonettes from said upstream end to said downstream end, the feed channel communicating with said upstream ends of said cyclonettes and feeding said grain-based liquid medium processing stream to said upstream ends thereof, and the outwardly-flowing channel communicating with and immersing said downstream ends of said cyclonettes in said grain-based liquid medium processing stream and conveying said grain-based liquid medium processing stream away from said downstream ends of said cyclonettes.

19. The method of claim 18 wherein the flow channel includes a first portion tapering inwardly in a downstream direction.

20. The method of claim 18 wherein the liquid treatment apparatus further comprises a throat portion of substantially constant internal diameter positioned upstream of said upstream end of said unidirectional flow channel.

21. The method of claim 18 wherein the liquid treatment apparatus further comprises a vortex finder received in each of said cyclonettes adjacent said upstream end thereof.

22. The method of claim 18 wherein the liquid treatment apparatus further comprises:
an orifice plate having an orifice defined therethrough positioned in each of said cyclonettes adjacent said upstream end of said unidirectional flow channel; and
said orifice having a diameter smaller than that of said flow channel at said upstream end thereof.

23. The method of claim 18 wherein the liquid treatment apparatus further comprises:
first and second, concentric, cylindrical casings defining therebetween said feed channel, and
said plurality of cyclonettes being mounted in said second cylindrical casing.

24. The method of claim 18 wherein the liquid treatment apparatus further comprises a passageway extending through a wall of each of said cyclonettes.

25. A system for alcohol production comprising:
a liquid treatment apparatus configured to apply hydraulic cavitation to a grain-based liquid medium processing stream at one or more locations in an alcohol production process; and
an alcohol production facility having the grain-based liquid medium processing stream in the alcohol production process, the alcohol production process adapted for use with the liquid treatment apparatus, wherein the liquid treatment apparatus applies hydraulic cavitation to the liquid medium processing stream at the one or more points in the alcohol production process by subjecting the grain-based liquid medium processing stream to shear under vacuum within the liquid treatment apparatus.

26. The method of claim 25 wherein the grain-based liquid medium processing stream is selected from the group consisting of corn, rye, sorghum, wheat, barley, oats, and rice.

27. The method of claim 25 wherein the alcohol production process is a dry grind, modified dry grind, or wet mill ethanol production process.
